(19) United States
(12) Patent Application Publication
(10) Pub. No.: US 2014/0096768 A1
(43) Pub. Date: Apr. 10, 2014

(54) RESPIRATORY MASK

(71) Applicant: GaleMed Xiamen CO., LTD., Xiamen City (CN)

(72) Inventor: Gary C.J. LEE, New Taipei City (TW)

(21) Appl. No.: 13/935,574

(22) Filed: Jul. 5, 2013

(30) Foreign Application Priority Data
Oct. 8, 2012 (CN) 201220512014.8

(51) Int. Cl. A61M 16/06; A61M 16/20; A61M 16/08

(52) U.S. Cl. CPC A61M 16/06; A61M 16/0816; A61M 16/208
USPC 128/202.27; 128/206.21

(57) ABSTRACT

A respiratory mask includes a mask body and an air inlet connector. The mask body is formed with an air inlet. The air inlet connector is integrally formed at the air inlet. The respiratory mask can be direct connected to an oxygen tube, an air storage bag, a mist spray tank, a medicine feed tank or a standard air tube of 22 mm, without the need for a separate connector. The respiratory mask decreases the number of the parts and is easy to operate for medical workers to change different pipes, without changing the respiratory mask.

### BACKGROUND OF THE INVENTION

1. Field of the Invention

The present invention relates to a mask, and more particularly to a respiratory mask.

2. Description of the Prior Art

For a serious patient, the patient needs to wear a respiratory mask to cover the mouth and the nose of the patient to assist the patient in breathing or in pouring medial gas along with breath so that the patient can maintain normal physiological functions. A conventional respiratory mask has a mask body. The mask body is formed with an air inlet for connecting an oxygen tube, an air storage bag, a mist spray tank, a medicine feed tank or a standard air tube of 22 mm. Because all the mask body, the oxygen tube, the air storage bag, the mist spray tank, the medicine feed tank and the standard air tube of 22 mm are made of soft materials so the is connection between the mask body and the oxygen tube, the air storage bag, the mist spray tank, the medicine feed tank or the standard air tube of 22 mm is not easy to operate. It is required to use a hard connector for connecting both. This connection is complicated and the connector is manufactured separately to increase the cost. In order to ensure the inputted gas has a certain oxygen and pressure, the connector is provided with a one-way inlet valve plate to prevent the outputted gas of the patient from entering the oxygen tube, the mist spray tank or other pipes. However, the inlet valve plate is mounted on the connector. The connector easily disengages from the air inlet to influence treatment. Accordingly, the inventor of the present invention has devoted himself based on his many years of practical experiences to solve these problems.

### SUMMARY OF THE INVENTION

The primary object of the present invention is to provide a respiratory mask which has a simple configuration and is easy to operate. The respiratory mask can be direct connected to an oxygen tube, an air storage bag, a mist spray tank, a medicine feed tank or a standard air tube of 22 mm, without the need for a separate connector. The present invention can lower the risk of disengagement of the parts and lower the manufacture cost.

Another object of the present invention is to provide a respiratory mask, which can prevent the outputted breath of the patient from entering the oxygen tube, the air storage bag, the mist spray tank, the medicine feed tank or the standard air tube of 22 mm to ensure consistent concentration and pressure of the delivered gas to enhance medial quality.

In order to achieve the aforesaid object, the respiratory mask of the present invention comprises a mask body and an air inlet connector. The mask body is formed with an air inlet. The air inlet connector is integrally formed at the air inlet.

Preferably, the air inlet connector is adapted to mate with a standard connector of an oxygen tube, a standard air tube of 22 mm, an air storage bag, a medicine feed tank or a mist spray tank.

Preferably, the inner side of the air inlet is provided with a one-way inlet valve plate.

Preferably, two sides of the mask body are respectively formed with an air outlet.

Accordingly, the air inlet connector of the respiratory mask of the present invention is integrally formed at the air inlet so that the respiratory mask can be direct connected with the oxygen tube, the air storage bag, the mist spray tank, the medicine feed tank or the standard air tube of 22 mm, without the need for a separate connector. The air inlet connector is adapted to mate with the standard connector of the oxygen tube, the air storage bag, the mist spray tank, the medicine feed tank or the standard air tube of 22 mm. The present invention is convenient for medical workers to change different pipes without changing the respiratory mask so it can be used widely. The inner side of the air inlet is provided with a one-way inlet valve plate. Two sides of the mask body are respectively formed with an air outlet. The air outlet is provided with an outlet valve plate so that the delivered gas is sent to the mask body in a one-way manner for medical use to the patient and the outputted breath of the patient is discharged to outside in a one-way manner through the air outlet. This can prevent the patient from inhaling external air to lower the concentration of oxygen. That is, the present invention can prevent the outputted is breath of the patient from entering the oxygen tube, the air storage bag, the mist spray tank, the medicine feed tank or the standard air tube of 22 mm to ensure consistent concentration and pressure of the delivered gas to enhance medial quality.

### BRIEF DESCRIPTION OF THE DRAWINGS

FIG. 1 is a perspective view according to a preferred embodiment of the present invention;

FIG. 2 is a top view according to the preferred embodiment of the present invention; and

FIG. 3 is a sectional view according to the preferred embodiment of the present invention.

### DETAILED DESCRIPTION OF THE PREFERRED EMBODIMENTS

Embodiments of the present invention will now be described, by way of example only, with reference to the accompanying drawings.

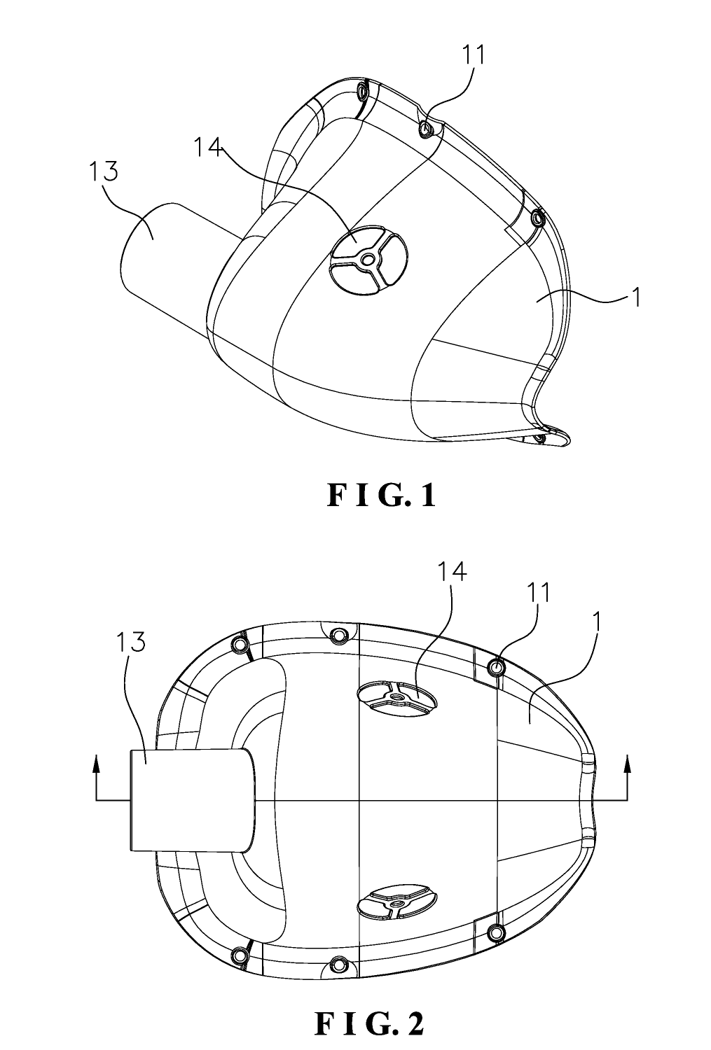

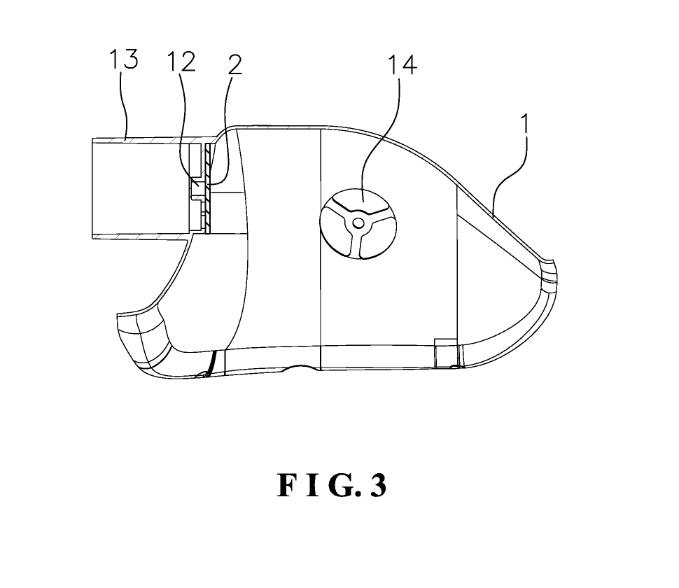

As shown in FIG. 1, FIG. 2 and FIG. 3, the present invention discloses a respiratory mask. The respiratory mask comprises a mask body 1 and an air inlet connector 13. The mask body 1 has a plurality of holes 11 at a circumferential portion thereof for connecting elastic straps, so that the mask body 1 can be attached to the face of a patient through the elastic straps. The patient can select the elastic straps in a two-way wearing manner as desired to position the mask on the head, or in a one-way wearing manner.

The mask body 1 is formed with an air inlet 12. The air inlet connector 13 is integrally formed at the air inlet 12. The air inlet connector 13 is adapted to mate with a standard connector of an oxygen tube, an air storage bag, a mist spray tank, a medicine feed tank or a standard air tube of 22 mm, so that the air inlet connector 13 is can be direct connected to the oxygen tube, the air storage bag, the mist spray tank, the medicine feed tank or the standard air tube of 22 mm, without the need for a separate connector. The present invention decreases the number of the parts and is easy to operate, namely, it can lower the risk of disengagement of the parts and lower the manufacture cost, it is convenient for medical workers to change different pipes without changing the respiratory mask so it can be used widely. The inner side of the air inlet 12 is provided with a one-way inlet valve plate 2. Two sides of the mask body 1 are respectively formed with an air outlet 14. The air outlet 14 is provided with an outlet valve plate so that the delivered gas is sent to the mask body 1 in a one-way manner for medical use to the patient and the outputted breath of the patient is discharged to outside in a one-way manner through the air outlet. This can prevent the patient from inhaling external air to lower the concentration of oxygen. That is, the present invention can prevent the outputted breath of the patient from entering the oxygen tube, the air storage bag, the mist spray tank, the medicine feed tank or the standard air tube of 22 mm to ensure consistent concentration and pressure of the delivered gas to enhance medial quality.

Although particular embodiments of the present invention have been described in detail for purposes of illustration, various modifications and enhancements may be made without departing from the spirit and scope of the present invention. Accordingly, the present invention is not to be limited except as by the appended to claims.

What is claimed is:

1. A respiratory mask, comprising a mask body and an air inlet connector, the mask body being formed with an air inlet, the air inlet connector being integrally formed at the air inlet.

2. The respiratory mask as claimed in claim 1, wherein the air inlet connector is adapted to mate with a standard connector of an oxygen tube, a standard air tube of 22 mm, an air storage bag, a medicine feed tank or a mist spray tank.

3. The respiratory mask as claimed in claim 1, wherein an inner side of the air inlet is provided with a one-way inlet valve plate.

4. The respiratory mask as claimed in claim 3, wherein two sides of the mask body are respectively formed with an air outlet.
